(19) United States
(12) Patent Application Publication
(10) Pub. No.: US 2025/0041563 A1
(43) Pub. Date: Feb. 6, 2025

(54) CATHETER SHAFT AND CATHETER DEVICE

(71) Applicant: Japan Lifeline Co., Ltd., Tokyo (JP)

(72) Inventors: Hiroki KAMIYAMA, Tokyo (JP); Atsuto KIMURA, Tokyo (JP); Tsubasa IMAI, Tokyo (JP)

(21) Appl. No.: 18/665,125

(22) Filed: May 15, 2024

(30) Foreign Application Priority Data
Jul. 31, 2023 (JP) 2023-124181

(51) Int. Cl. A61M 25/00; A61M 25/10

(52) U.S. Cl. CPC A61M 25/005; A61M 25/0032; A61M 2025/0037; A61M 25/1018; A61M 2205/70

(57) ABSTRACT

A catheter shaft includes a shaft main body having a multi-lumen structure, and at least one reinforcement layer embedded in the shaft main body. When, during a tensile test of pulling a test piece obtained from the catheter shaft under test conditions of a 30-mm distance between chucks and a tensile speed of 300 mm/min, a percentage of change amount in a minimum outer diameter of the test piece after pulling to the minimum outer diameter before pulling at a predetermined position of the test piece is defined as a diameter reduction percentage (%), and a percentage of change amount in an axial-direction dimension of the catheter shaft after pulling to the axial-direction dimension before pulling is defined as an elongation percentage (%), the diameter reduction percentage is 5% or greater when the elongation percentage reaches 100% without breakage of the test piece in the tensile test.

### CROSS REFERENCE TO RELATED APPLICATIONS

This application claims the benefit of priority to Japanese Patent Application Number 2023-124181 filed on Jul. 31, 2023. The entire contents of the above-identified application are hereby incorporated by reference.

### TECHNICAL FIELD

The present disclosure relates to a catheter device.

### BACKGROUND

JP 2018-171372 A discloses a catheter device including a catheter shaft. This catheter shaft includes a shaft main body having a multi-lumen structure and a reinforcement layer to be embedded in the shaft main body.

### SUMMARY

A catheter shaft may be inserted from a peripheral blood vessel in the hand, foot, or the like. When a catheter shaft is passed through a small blood vessel such as peripheral blood vessel, abnormal vasoconstriction called a spasm may occur in the blood vessel. When an external force is applied to the catheter shaft from a blood vessel due to this spasm, there is a problem in that the catheter shaft cannot be pulled out even if pulled toward outside the body (proximal side).

One object of the present disclosure is to provide a catheter shaft that is easily pulled out of the body and is unlikely to break when pulled out, even when an external force is applied from a blood vessel due to a spasm.

A catheter shaft according to an aspect of the present disclosure includes a shaft main body having a multi-lumen structure, and at least one reinforcement layer embedded in the shaft main body. When, during a tensile test of pulling a test piece obtained from the catheter shaft under test conditions of a 30-mm distance between chucks and a tensile speed of 300 mm/min, a percentage of change amount in a minimum outer diameter of the test piece after pulling to the minimum outer diameter before pulling at a predetermined position of the test piece is defined as a diameter reduction percentage (%), and a percentage of change amount in an axial-direction dimension of the catheter shaft after pulling to the axial-direction dimension before pulling is defined as an elongation percentage (%), the diameter reduction percentage is 5% or greater when the elongation percentage reaches 100% without breakage of the test piece in the tensile test.

A catheter shaft according to another aspect of the present disclosure includes a shaft main body having a multi-lumen structure, and at least one reinforcement layer embedded in the shaft main body. When, during a tensile test of pulling a test piece obtained from the catheter shaft under test conditions of a 30-mm distance between chucks and a tensile speed of 300 mm/min, a percentage of change amount in a minimum outer diameter of the test piece after pulling to the minimum outer diameter before pulling at a predetermined position of the test piece is defined as a diameter reduction percentage (%), the diameter reduction percentage is 20% or greater when application of a tensile load of 90 N to the test piece is started in the tensile test.

According to the present disclosure, it is possible to provide a catheter shaft that is easily pulled out of the body and is unlikely to break when pulled out, even when an external force is applied from a blood vessel due to a spasm.

### BRIEF DESCRIPTION OF DRAWINGS

FIG. 1A is an explanatory view illustrating a state before elongation deformation of a braid, and FIG. 1B is an explanatory view illustrating a state after elongation deformation of the braid.

FIG. 2 is an overall view illustrating a catheter device of an embodiment.

FIG. 3 is a cross-sectional view along A-A in FIG. 2.

FIG. 4A is a diagram illustrating an example of a braiding pattern of the braid of the embodiment, and FIG. 4B is a diagram illustrating another example of the braiding pattern.

FIG. 5A is a schematic view illustrating a test piece used for a tensile test, and FIG. 5B is a schematic view illustrating a state of the test piece set in a tensile tester.

FIG. 6 is a graph showing a relationship between an elongation percentage and a diameter reduction percentage obtained by the tensile test.

FIG. 7 is a graph showing a relationship between a tensile load and a diameter reduction percentage obtained by the tensile test.

### DESCRIPTION OF EMBODIMENTS

Hereinafter, embodiments for carrying out the present disclosure will be described. The same or equivalent constituent elements are denoted by the same reference signs, and redundant descriptions are omitted. In the drawings, for convenience of description, components are omitted, enlarged, or reduced, as appropriate. The drawings are to be viewed in accordance with the orientation of the reference signs. As used herein, “or” means that at least one of two or more of the elements is adopted.

First, the background that led to the conception of the catheter shaft of the present disclosure will be described. The catheter shaft assumed includes a shaft main body having a multi-lumen structure and at least one reinforcement layer embedded in the shaft main body. The inventors of the present application proceeded with studies of such a catheter shaft for facilitating pullout of the catheter shaft to outside the body when an external force is applied from a blood vessel due to a spasm.

The reason for focusing on the multi-lumen structure is that, in the case of the multi-lumen structure, when the catheter shaft to which an external force is applied from a blood vessel due to a spasm is pulled out, the catheter shaft easily breaks as compared with a single-lumen structure. The cause is not quite clear, but the following is conceivable. In the case of the multi-lumen structure, an area having a thin thickness is likely to be formed between an outer peripheral surface of the shaft main body and the lumens or between adjacent lumens as compared with the single-lumen structure. In the case of the multi-lumen structure, as compared with the single lumen structure, conceivably the catheter shaft easily breaks due to stress concentration generated in the area having a thin thickness.

In the course of this study, the inventors of the present application newly focused on ease of thinning when the catheter shaft, to which an external force is applied from a blood vessel due to a spasm, is pulled in relation to ease of pullout of the catheter shaft. This is because, when the catheter shaft is pulled, the catheter shaft is thinned at the location where the external force is applied from the blood vessel, facilitating pullout of the catheter shaft.

The inventors of the present application discovered the concept of a diameter reduction percentage of the catheter shaft measurable by a tensile test as a new index indicating this ease of thinning of the catheter shaft. As a result of confirmation of the diameter reduction percentage that can be acquired in a catheter shaft having a typical multi-lumen structure, it was newly discovered that a maximum diameter reduction percentage that can be acquired in this test piece is usually about 0 to 3% and does not exceed 5%.

Therefore, in relation to the problem of facilitating pullout of the catheter shaft, the catheter shaft of the present disclosure, in consideration of ease of measurement of the diameter reduction percentage, satisfies at least one of the following first shaft condition or second shaft condition in relation to the shaft. The first shaft condition is that the diameter reduction percentage is 5% or greater when an elongation percentage (described below) of the test piece reaches 100% without breakage of the test piece in a tensile test. The second shaft condition is that the diameter reduction percentage is 20% when application of a tensile load of 90 N to the test piece is started without breakage of the test piece in the tensile test. Here, the elongation percentage of the test piece is a value indicating the ease of elongation of the catheter shaft. Both shaft conditions are under the condition that, at the time of the tensile test, the diameter reduction percentage to be measured when the reference condition (elongation percentage of the test piece reaches 100% or start of application of the tensile load of 90 N to the test piece) is satisfied is an allowable value or greater. This allowable value of the diameter reduction percentage is set as a guide in relation to the problem of facilitating pullout of the catheter shaft. With at least one of the first shaft condition or the second shaft condition being satisfied, the catheter shaft can be easily pulled out and is less likely to break when pulled out, even when an external force is applied from a blood vessel due to a spasm, as compared with a case in which the condition is not satisfied.

Further, the inventors of the present application newly discovered the presence of an advantageous tendency that the acquirable maximum diameter reduction percentage of the test piece is increased as the acquirable maximum elongation percentage of the test piece is increased. As a result of confirmation of the relationship between the diameter reduction percentage and the elongation percentage of a catheter shaft having a typical multi-lumen structure, it was newly discovered that, when the maximum diameter reduction percentage is about 0 to 3%, the maximum elongation percentage is 50% or less and does not exceed 100%. The concept for increasing the acquirable elongation percentage of the test piece will now be described.

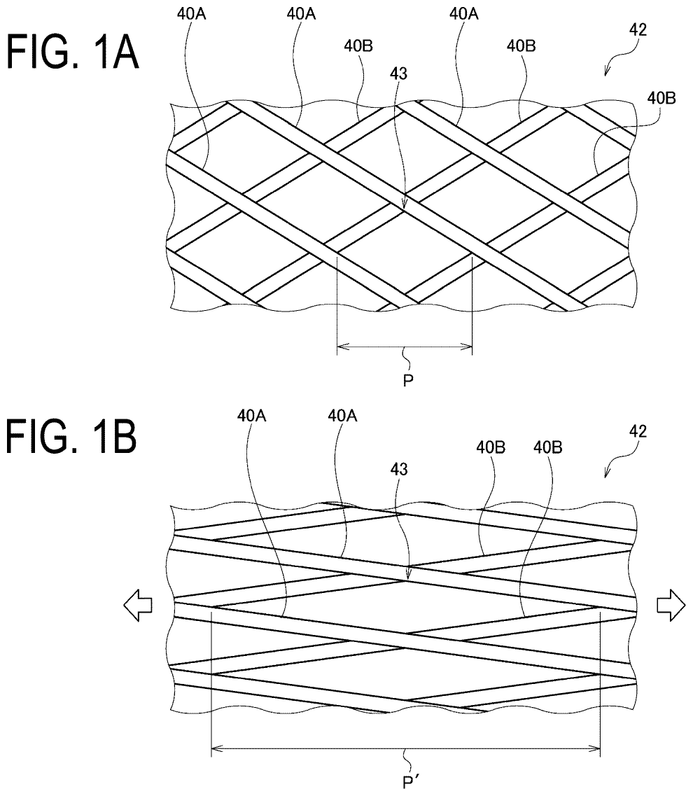

FIG. 1A and FIG. 1B are views illustrating states before and after elongation deformation of a braid 42 constituting a reinforcement layer of a catheter shaft. In FIG. 1A, an axial-direction dimension of a mesh 43 of the braid 42 is P and, in FIG. 1B, the axial-direction dimension of the mesh 43 is extended to P′. The inventors of the present application discovered that, in order to increase the elongation percentage described above when the reinforcement layer is constituted by the braid 42, it is important to focus not only on ease of elongation of the shaft main body, but also on ease of movement of element wires 40A, 40B at the time of elongation deformation of the braid 42. This ease of movement of the element wires 40A, 40B improves as a friction force at intersection points between the element wires 40A, 40B decreases or as a pitch of the element wires 40A, 40B decreases. An elongation amount (hereinafter referred to as a “limit elongation amount”) to which a shaft main body made of resin can be elongated until breakage is usually significantly great compared with an elongation amount to which the braid 42 can be elongated until breakage. However, in a case in which the braid 42 is embedded in the shaft main body, when the ease of movement of the element wires 40A, 40B is excessively low, a problem arises that the elongation deformation of the shaft main body is restrained by the braid 42 at a timing excessively earlier than the timing at which the shaft main body reaches the limit elongation amount. When the elongation deformation of the shaft main body is restricted by the braid 42 as described above, the elongation deformation amount of the entire test piece becomes insufficient, which is disadvantageous in acquiring a great elongation percentage.

Here, the inventors of the present application discovered that, in order to increase the acquirable elongation percentage of the test piece, it is effective to appropriately increase the ease of movement of the element wires 40A, 40B by adjusting factors that affect the ease of movement of the element wires 40A, 40B when the braid 42 is about to undergo elongation deformation. By appropriately increasing the ease of movement of the element wires 40A, 40B, it is possible to avoid a situation in which the elongation deformation of the shaft main body is restrained by the braid 42 at a timing excessively earlier than when the limit elongation amount of the shaft main body is reached, which is advantageous in acquiring a great elongation percentage. Hereinafter, the catheter shaft conceived on the basis of such concepts will be described in detail.

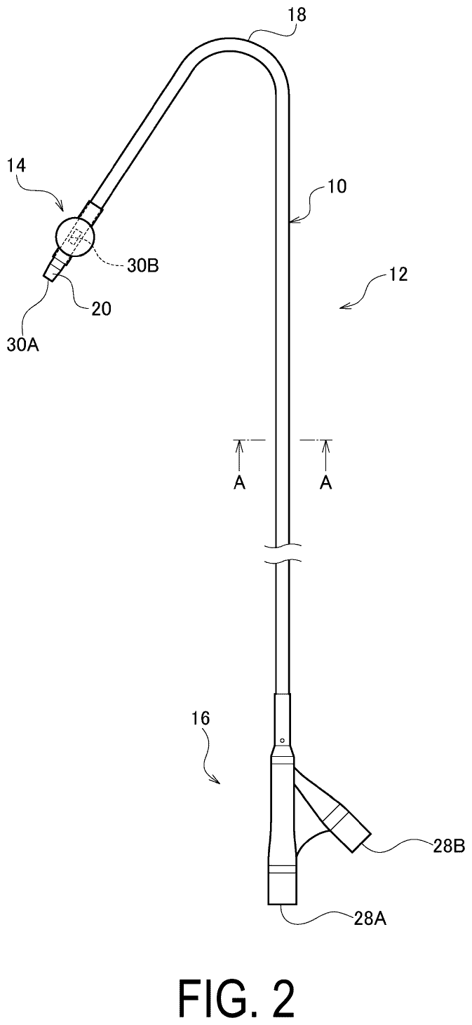

Description will be made with reference to FIG. 2. Hereinafter, when a positional relationship of each component of a catheter device 12 is described, description will be made using an axial direction, a radial direction, and a circumferential direction of a catheter shaft 10, and these will simply be referred to as “axial direction,” “radial direction,” and “circumferential direction.”

The catheter shaft 10 is used as a portion of the catheter device 12. The catheter device 12 of the present embodiment is a balloon catheter in which a balloon 14 is mounted on the catheter shaft 10. Specific examples of the catheter device 12 are not particularly limited. For example, the catheter device 12 may be used as an electrode catheter, a delivery catheter, a guiding catheter, a foreign substance removal catheter, an imaging catheter, or a micro-catheter. The catheter shaft 10 is inserted into a body for treatment of a target organ, such as a blood vessel. “Treatment” here refers to an act related to treatment or examination of a living body. The catheter shaft 10 is inserted into the body from a peripheral blood vessel in the hand or foot, for example.

In addition to the catheter shaft 10, the catheter device 12 includes a handle 16 that is attached to a proximal end portion of the catheter shaft 10 and is grasped by a practitioner. As an optional configuration, the catheter device 12 of the present embodiment includes the balloon 14 to be attached to the distal end portion of the catheter shaft 10.

The catheter shaft 10 is flexible enough to be bent and deformed. Herein, an example is illustrated in which a bending portion 18 is provided at an intermediate portion in the axial direction of the catheter shaft 10. A tip member 20 is attached to the distal end portion of the catheter shaft 10. An outer diameter (diameter) of the catheter shaft 10 is, for example, 5 Fr (1. 67 mm) to 9.5 Fr (3.2 mm).

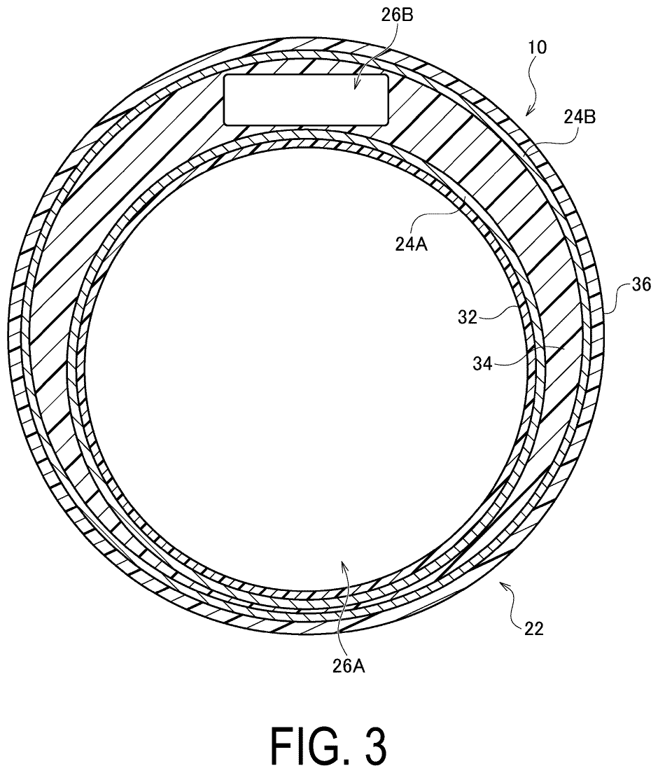

Description will now be made with reference to FIG. 3. FIG. 3 illustrates an entire range in which reinforcement layers 24A, 24B described below are present as viewed from the axial direction. Actually, in a cross section orthogonal to the axial direction, element wires (described below) partially constituting the reinforcement layers 24A, 24B are present in this range illustrating the reinforcement layers 24A, 24B.

The catheter shaft 10 includes a shaft main body 22 having a multi-lumen structure and at least one reinforcement layer 24A, 24B embedded in the shaft main body 22. Here, the multi-lumen structure refers to a structure in which a plurality of lumens 26A, 26B are formed in the shaft main body 22. Although an example in which the quantity of the lumens 26A, 26B is two is illustrated here, the quantity may be three or more. The plurality of lumens 26A, 26B of the present embodiment include the main lumen 26A and the sub-lumen 26B. In the present embodiment, in a cross section of the catheter shaft 10 orthogonal to the axial direction, a cross-sectional area of the main lumen 26A is larger than a cross-sectional area of the sub-lumen 26B. A size relationship between the cross-sectional areas of the lumens is not particularly limited, and the cross-sectional areas of all lumens may be the same.

Each of the plurality of lumens 26A, 26B is used for passage of at least one of a medical device or a fluid. The main lumen 26A of the present embodiment is used for passage of a medical device. The sub-lumen 26B of the present embodiment is used for passage of a fluid that expands the balloon 14. The medical device to be passed through the lumens 26A, 26B refers to, for example, a guide wire, a stent graft, a coil, a wire member, as well as various catheters such as an electrode catheter, a balloon catheter, and a micro-catheter. The wiring member as used herein refers to, for example, a wiring flexible substrate to be electrically connected to an electric device to be attached to the catheter shaft 10. The electric device as used herein is, for example, an ultrasonic transducer used in intracardiac echocardiography (ICE). The fluid to be passed through the lumens 26A, 26B refers to, for example, a liquid agent such as a contrast agent and a liquid for irrigation (saline or the like) in addition to the fluid for expanding the balloon 14.

An object to be passed through the lumens 26A, 26B is introduced into the lumens 26A, 26B from outside the body through introduction ports 28A, 28B provided in the handle 16, and is guided outside of the lumens 26A, 26B through outlet ports 30A, 30B provided in the catheter shaft 10. FIG. 2 illustrates the first introduction port 28A and the first outlet port 30A corresponding to the main lumen 26A, and the second introduction port 28B and the second outlet port 30B corresponding to the sub-lumen 26B.

The shaft main body 22 includes at least one resin layer. The shaft main body 22 of the present embodiment includes, as such resin layers, an inner layer 32, an intermediate layer 34 covering the inner layer 32, and an outer layer 36 covering the intermediate layer 34. The main lumen 26A is formed by the inner layer 32, and the sub-lumen 26B is formed by the intermediate layer 34.

The reinforcement layers 24A, 24B are used to reinforce the shaft main body 22. The reinforcement layers 24A, 24B of the present embodiment include the inner reinforcement layer 24A surrounding the main lumen 26A and the outer reinforcement layer 24B surrounding the main lumen 26A and the sub-lumen 26B.

Note that the number of resin layers constituting the shaft main body 22 and the number of reinforcement layers to be embedded in the shaft main body 22 are not particularly limited. Further, positions of the reinforcement layer and the lumens in the shaft main body 22 are not particularly limited as well. For example, only one of the inner reinforcement layer 24A and the outer reinforcement layer 24B described above may be present as the reinforcement layer, or a reinforcement layer different from these may be present.

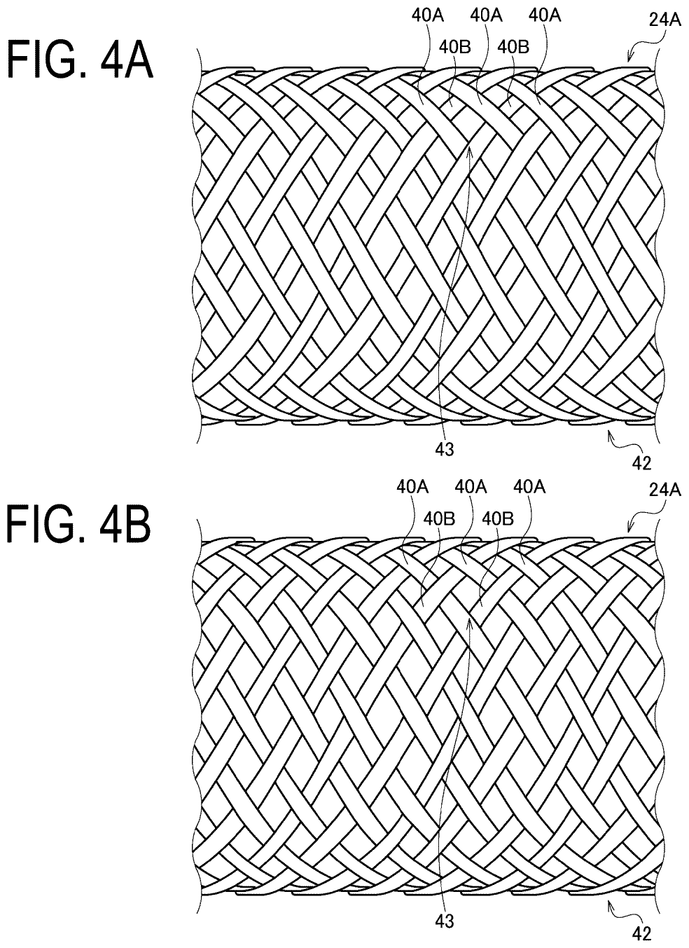

Description will now be made with reference to FIG. 4A and FIG. 4B. FIG. 4A and FIG. 4B are cross-sectional views of a portion of the braid 42 constituting the reinforcement layer 24A as viewed from an outer side in the radial direction. The reinforcement layers 24A, 24B of the present embodiment are the braids 42 formed by braiding the plurality of element wires 40A, 40B into a tubular and net-like shape. The reinforcement layers 24A, 24B may be constituted by coils formed of a helical element wires instead of the braids 42.

The braid 42 is constituted by the plurality of first element wires 40A extending toward one side in the circumferential direction toward one side in the axial direction, and the plurality of second element wires 40B extending toward the other side in the circumferential direction toward one side in the axial direction. The plurality of first element wires 40A and the plurality of second element wires 40B intersect each other. The braid 42 forms a plurality of the meshes 43 by the plurality of element wires 40A, 40B. The meshes 43 are formed as gaps at locations surrounded by the first element wires 40A adjacent to each other in the circumferential direction and the second element wires 40B adjacent to each other in the circumferential direction, and have rhombic shapes.

The braid 42 is braided in a braiding pattern of repeating a braiding unit in which the first element wire 40A and the second element wire 40B are each placed over and under the other element wire in the axial direction thereof. Examples of this braiding pattern include a half pattern (two over two under pattern) illustrated in FIG. 4A and a full pattern (one over one under pattern) illustrated in FIG. 4B. The half pattern of FIG. 4A is a braiding pattern of repeating a braiding unit in which the first element wire 40A sequentially passes over an outer side of two second element wires 40B in the radial direction and then an inner side of two second element wires 40B in the radial direction. The full pattern of FIG. 4B is a braiding pattern of repeating a braiding unit in which the first element wire 40A sequentially passes over an outer side of the second element wire 40B in the radial direction and then an inner side of the second element wire 40B in the radial direction. A number of other element wires (for example, the second element wire 40B) intersecting one element wire (for example, the first element wire 40A) per braiding unit is referred to as the number of element wire intersections per braiding unit. The number of element wire intersections per braiding unit of the half pattern is four, and the number of element wire intersections per braiding unit of the full pattern is two. The braiding patterns of the braid 42 mentioned here are merely examples, and various other braiding patterns may be adopted.

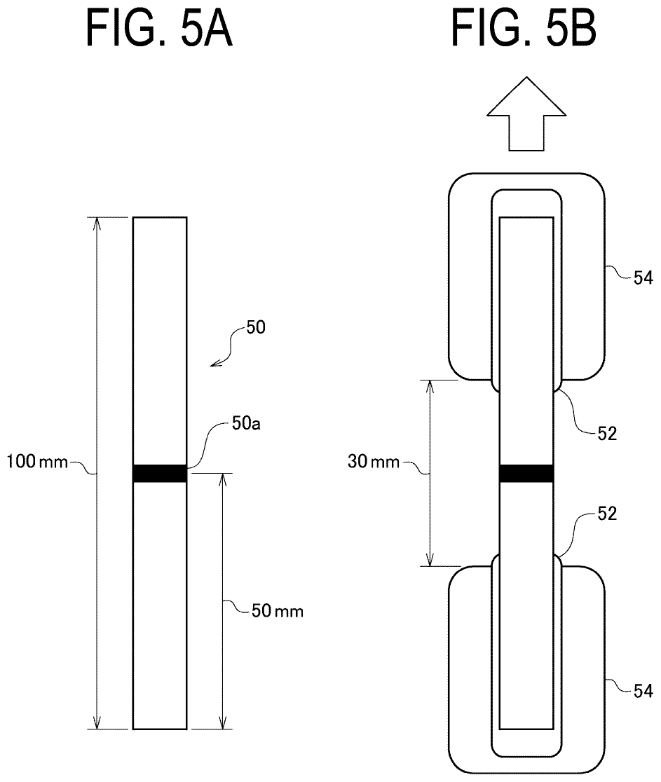

Next, a tensile test used for measuring the elongation percentage and the diameter reduction percentage of the catheter shaft 10 described above will be described. Description will be made with reference to FIG. 5A. In this tensile test, a test piece 50 obtained by cutting the catheter shaft 10 to a dimension of 100 mm in the axial direction is used. In this test piece 50, a marked line portion 50a is provided by drawing a line with a magic marker at a central position in the axial direction (an axial-direction position at 50 mm from an axial-direction end).

Prior to the tensile test, an outer diameter of the marked line portion 50a of the test piece 50 before being pulled is measured by a non-contact type outer diameter measurement device. At this time, a minimum outer diameter (diameter) of the marked line portion 50a of the test piece 50 in a cross section orthogonal to the axial direction is measured. The term “minimum outer diameter” as used herein refers to a minimum value of measured values obtained when the outer diameter of the marked line portion 50a of the test piece 50 is measured by an outer diameter measurement device across the entire area.

Description will now be made with reference to FIG. 5B. The tensile test is performed so as to satisfy the items listed under “Conditions” below. For example, Model No. EZ Test EZ-LX manufactured by Shimadzu Corporation is used as the tensile tester, and Model No. PFG-5kNA is used as the air chuck. Specific examples of the tensile tester and the air chuck are not limited as long as the devices satisfy the conditions below. The test piece 50 pulled by the tensile test may be uniformly reduced in a circular shape or may be reduced in a flat shape.

### Conditions

Use a tensile tester having a load cell rated capacity of 5 kN
Cover both end portions of the test piece 50 with silicone tubes 52 (inner diameter 3 mm, outer diameter 5 mm) to prevent damage due to direct contact between the air chucks 54 and the test piece 50 (the silicone tube 52 may protrude from the air chucks 54)
Chuck both upper and lower end portions of the test piece 50 by the pair of air chucks 54 having a maximum capacity of 5 kN at a chucking pressure of 0.6 MPa so that the silicone tubes 52 do not move
Pull the test piece 50 by moving one of the air chucks 54 under test conditions of a chuck-to-chuck distance that is a distance between the pair of air chucks 54 being 30 mm and a tensile speed being 300 mm/min

This tensile test is ended when the test piece 50 is pulled until a predetermined test end condition is satisfied. The test end condition herein is that the elongation percentage (described below) of the test piece 50 reaches a specified elongation percentage (herein, 100%) without breakage of the test piece 50. In a case in which the test piece 50 breaks before the test end condition is satisfied, the tensile test is ended. When the tensile test is ended, the tensile load to be applied to the test piece 50 is removed, and the test piece 50 is removed from the tensile tester. Subsequently, the diameter reduction percentage, described below, of the test piece 50 is measured using the test piece 50 subjected to the tensile test. In addition, the presence or absence of exposure of the reinforcement layers 24A, 24B is determined for the test piece 50 pulled until the specified elongation percentage is reached without breakage of the test piece 50.

The presence or absence of breakage of the test piece 50 is visually determined. “The test piece 50 breaks” means that portions on both sides of the test piece 50 in the axial direction are not connected to each other and are completely separated from each other in any of the shaft main body 22 and the reinforcement layers 24A, 24B of the test piece 50.

The axial-direction dimension of the test piece 50 before pulling is set to L0 (mm), the axial-direction dimension after pulling is set to L1 (mm), and the amount of change in the axial-direction dimension after pulling is set to ΔL (=| L1−L0|). This amount of change ΔL in the axial-direction dimension is represented by the amount of movement of one air chuck 54 in the tensile test described above. At this time, the elongation percentage (%) of the test piece 50 is the percentage of the amount of change ΔL in the axial-direction dimension of the test piece 50 after pulling to the axial-direction dimension L0 (mm) before pulling, and is expressed by the following equation (1). As can be understood from the following equation (1), the elongation percentage is a value that can be sequentially obtained from the movement amount (ΔL) of the air chuck 54 during the tensile test.

$$Elongation percentage ( \% ) = ( Δ L / L 0 ) \times 100 \qquad ( 1 )$$

The minimum outer diameter (diameter) at a predetermined position of the test piece 50 before pulling is set to Φ0 (mm), the minimum outer diameter (diameter) at the predetermined position after pulling is set to 1 (mm), and the amount of change in the minimum outer diameter after pulling is set to ΔΦ(=(Φ0−Φ1). At this time, the diameter reduction percentage (%) of the test piece 50 is the percentage of the amount of change ΔΦ in the minimum outer diameter after pulling to the minimum outer diameter Φ0 before pulling, and is expressed by the following equation (2). Here, the predetermined position refers to the center position in the axial direction where the marked line portion 50a of the test piece 50 described above is located.

$$Diameter reduction percentage ( \% ) = ( ΔΦ / Φ0 ) \times 100 \qquad ( 2 )$$

The presence or absence of exposure of the reinforcement layers 24A, 24B is determined by observing an axial-direction range of the marked line portion 50a ±10 mm with a microscope at a magnification of 50 times. In this specification, “the reinforcement layers 24A, 24B are exposed to the outside” means that the shaft main body 22 of the test piece 50 is partially torn, exposing the reinforcement layers 24A, 24B to the external space. To satisfy the condition that “the reinforcement layer is exposed to the outside,” in a case in which there are a plurality of reinforcement layers 24A, 24B, at least one of the reinforcement layers 24A, 24B need only be exposed to the outside. To satisfy the condition that “the reinforcement layer is exposed to the outside,” only the inner reinforcement layer 24A may be exposed to the outside, only the outer reinforcement layer 24B may be exposed to the outside, or both the inner reinforcement layer 24A and the outer reinforcement layer 24B may be exposed to the outside.

The diameter reduction percentage when the test piece 50 satisfies the reference conditions described above is an indicator indicating the ease of thinning when the catheter shaft 10 to which an external force is applied from a blood vessel due to a spasm is pulled. This diameter reduction percentage is proportional to the ease at which the catheter shaft 10, in a state in which an external force is applied from a blood vessel, is thinned in the contracted blood vessel by the catheter shaft 10 being pulled toward outside the body (proximal side) and to the ease at which the catheter shaft 10 is pulled out from the blood vessel. From such a viewpoint, the catheter shaft 10 is subject to the condition that the diameter reduction percentage when the elongation percentage of the test piece 50 reaches 100% without breakage of the test piece 50 (when the first reference condition is satisfied) is 5% or greater. From the same viewpoint, in the catheter shaft 10, the diameter reduction percentage when the elongation percentage similarly reaches 100% is preferably 10% or greater, and more preferably 15% or greater. A lower limit of the allowable diameter reduction percentage as a shaft to which the disclosure is applied is specified by using an approximate value, the value being great in the catheter shaft 10 having a multi-lumen structure as compared with 0 to 3%, which is the normal value. An upper limit of the diameter reduction percentage when the elongation percentage of the test piece 50 reaches 100% without breakage of the test piece 50 is not particularly limited, but is realistically 80%.

Further, the catheter shaft 10 is subject to the condition that the elongation percentage of the test piece 50 is 100% without breakage of the test piece 50. This makes it possible to facilitate pullout of the catheter shaft 10 while making breakage less likely at the time of pullout.

The catheter shaft 10 is subject to the condition that the reinforcement layers 24A, 24B are not exposed to the outside when the elongation percentage of the test piece 50 reaches 100% in the tensile test as described above. Accordingly, in the process of pulling out the catheter shaft 10 to which an external force is applied from a blood vessel, the reinforcement layers 24A, 24B are not exposed from the shaft main body 22 until the elongation percentage of the test piece 50 reaches 100%. This makes it possible to reduce the risk that the reinforcement layers 24A, 24B exposed are rubbed against living tissue.

The elongation percentage acquirable by the test piece 50 has an advantageous tendency that, as this value increases, the diameter reduction percentage acquirable by the test piece 50 is increased. From such a viewpoint, a lower limit of the elongation percentage allowed for a catheter shaft to which the disclosure is applied is increasingly preferable as the elongation percentage increases in increments of 100% from 100%. That is, this lower limit of the elongation percentage is preferably 200%, 300%, 400%, and 500%, in this order. An upper limit of the elongation percentage is not particularly limited, but is realistically 1000%. In any case, it is assumed that the test piece 50 is not broken.

In terms of increasing the elongation percentage without breakage of the test piece 50, in a case in which the braid 42 is adopted as the reinforcement layers 24A, 24B, it is preferable to adjust factors that affect the ease of movement of the element wires 40A, 40B when the braid 42 is about to undergo elongation deformation, based on the assumption that materials of the shaft main body 22 having an ease of elongation that can achieve the elongation percentage are selected. As described above, the ease of movement of the element wires 40A, 40B improves as the friction force at the intersection points between the element wires 40A, 40B decreases or as the pitch of the element wires 40A, 40B decreases.

The present inventors newly discovered that factors affecting the ease of movement of the element wires 40A, 40B include (1) cross-sectional shapes of the element wires 40A, 40B, (2) the quantity of the element wires 40A, 40B, (3) the braiding pattern of the braid 42, (4) a surface roughness and a friction coefficient of the element wires 40A, 40B, (5) the pitch of the element wires 40A, 40B, and the like. Among these factors, (1) to (4) are factors related to the friction force at the intersection points between the element wires 40A, 40B.

The cross-sectional shapes of the element wires 40A, 40B of (1) affect the contact area at the intersection points between the element wires 40A, 40B and, in terms of reducing the friction force at the intersection points, cross-sectional shapes capable of reducing the contact area are preferable. From this viewpoint, as the cross-sectional shapes of the element wires 40A, 40B, in terms of increasing the ease of movement of the element wires 40A, 40B, using a circular cross section, which has a small contact area at the intersection point, is more advantageous than using a flat cross section, which has a large contact area.

The quantity of the element wires 40A, 40B (quantity of wires constituting the element wires 40A, 40B) of (2) also affects the contact area at the intersection points between the element wires 40A, 40B and, in terms of reducing the friction force at the intersection points, a less quantity is more preferable. From this viewpoint, in terms of increasing the ease of movement of the element wires 40A, 40B, a quantity of one is more advantageous than a quantity that is a plurality.

The braiding pattern of the braid 42 of (3) affects the normal force at the intersection points between the element wires 40A, 40B and, in terms of reducing the friction force at the intersection points, a braiding pattern in which the number of intersection points of the element wires per braiding unit is great is preferable. From this viewpoint, as the braiding pattern of the braid 42, in terms of increasing the ease of movement of the element wires 40A, 40B, it is more advantageous to use a braiding pattern (for example, a half pattern) having a great number of element wire intersection points per braiding unit than to use a braiding pattern (for example, a full pattern) having a less number of element wire intersection points.

In terms of reducing the friction force at the intersection points between the element wires 40A, 40B, the element wires 40A, 40B may be subjected to a surface treatment for reducing any one of the surface roughness and the friction coefficient of the element wires 40A, 40B of (4).

In terms of increasing the ease of movement of the element wires 40A, 40B, preferably an inclination angle of the element wires 40A, 40B with respect to the axial direction is made as steep as possible. From this viewpoint, in terms of increasing the ease of movement of the element wires 40A, 40B, the pitch of the element wires 40A, 40B of (5) is more advantageous when narrow than when wide. The term pitch as used herein refers to an axial-direction dimension P (refer to FIGS. 1A and 1B) of the mesh 43 of the braid 42.

In terms of adjusting the elongation percentage without breakage of the test piece, at least any one of these factors (1) to (5) may be adjusted. In other words, it can be said that any one of these factors is adjusted so that the catheter shaft 10 satisfies the condition of the elongation percentage described above (for example, the condition of 100% or greater).

Note that, in a case in which coils are used as the reinforcement layers 24A, 24B, in terms of adjusting the elongation percentage, a coil pitch (pitch angle), a wire diameter of the element wire, a quantity, a raw material, a cross-sectional shape, and the like may be adjusted. For example, in terms of increasing the elongation percentage, the coil pitch may be decreased, the wire diameter may be narrowed, or the quantity may be decreased so as to facilitate elongation deformation of the element wires in the axial direction. In addition, in terms of increasing the elongation percentage, the material and the cross-sectional shape may be adjusted so as to facilitate elongation deformation of the element wires in the axial direction.

Examples of materials of the shaft main body 22 having an ease of elongation capable of achieving the elongation percentage described above include resins such as polytetrafluoroethylene (PTFE), perfluoroalkoxy fluororesin (PFA), polyetherblockamide (PEBAX; trade name), polyamide 12 (PA12), and thermo-plastic polyurethane (TPU). In terms of increasing the elongation percentage, preferably a material that easily elongates is used as the material of the shaft main body 22. Although the materials of the element wires 40A, 40B are not particularly limited, metals such as stainless steel and an Ni—Ti alloy may be used, for example. Further, a resin may be adopted as the raw material for the element wires 40A, 40B.

Note that, in terms of adjusting the diameter reduction percentage, the elongation percentage itself may be adjusted, or elements that affect the elongation percentage (factors affecting the ease of elongation of the raw material of the shaft main body 22 and the ease of movement of the element wires 40A, 40B) may be adjusted. For example, in terms of increasing the diameter reduction percentage, the adjustment method adopted for increasing the elongation percentage described above may be adopted. Further, in terms of increasing the diameter reduction percentage and the elongation percentage without exposing the reinforcement layers 24A, 24B to the outside, each of the following (1) to (3) may be adjusted within a range allowed by the design: (1) the raw material of the shaft main body 22, (2) a thickness of an area (outer layer 36 in the embodiment) of the shaft main body 22 on an outer side of the reinforcement layers 24A, 24B in the radial direction, and (3) factors that affect the ease of movement of the element wires 40A, 40B. For example, as a material of the shaft main body 22 of (1), preferably a material that easily elongates is adopted. In terms of adopting such a material that easily elongates, preferably a resin material having a low hardness is adopted because a resin material more easily elongates with a lower hardness. For example, PEBAX having a low hardness elongates more easily than PEBAX having a high hardness, and is thus preferable as the material of the shaft main body 22. Further, an inorganic substance such as barium sulfate may be added to the material of the shaft main body 22 for the purpose of improving radiopacity and the like. With a greater added amount of the inorganic substance, the shaft main body 22 is less likely to elongate. Accordingly, in terms of adopting a material that easily elongates, preferably the added amount of the inorganic substance is reduced to the extent possible or a material to which no inorganic substance is added is adopted. Increasing a partial thickness of the shaft main body 22 of (2) is advantageous in terms of avoiding exposure of the reinforcement layers 24A, 24B caused by partial tearing of the shaft main body 22. Adjusting factors that affect the ease of movement of the element wires 40A, 40B of (3) facilitates movement of the element wires 40A, 40B. If the element wires 40A, 40B do not readily move during the elongation deformation of the shaft main body 22, the elongation deformation of the shaft main body 22 is restrained by the braid 42 at an early stage, and the shaft main body 22 may be partially torn starting from the point of contact between the shaft main body 22 and the braid 42, as described above. Facilitating movement of the element wires 40A, 40B is advantageous in avoiding a situation in which the elongation deformation of the shaft main body 22 is restrained at an early stage by the braid 42 as described above, and avoiding exposure of the reinforcement layers 24A, 24B caused by partial tearing of the shaft main body 22.

A maximum load (N) that can be applied to the test piece 50 until the test piece 50 breaks in the tensile test is preferably 90 N or greater. This “maximum load” refers to the maximum tensile load that can be applied to the test piece 50 from the start of the pulling of the test piece 50 until the test piece 50 breaks. The inventors of the present application discovered that, when the practitioner applies a tensile load to the catheter shaft 10, to which an external force is applied from a blood vessel, by pulling the catheter shaft, the tensile load is usually less than the 90 N. By satisfying the condition of the maximum load described above, as long as the tensile load is within the range of the tensile load usually applied by a practitioner, it is possible to avoid a situation in which the catheter shaft 10 breaks.

A tensile amount (amount of change in the axial-direction dimension of the test piece 50 before and after pulling) of the test piece 50 when the test piece 50 is at the maximum load is referred to as a tensile amount at maximum load. At this time, there is no particular limitation on how the tensile load to be applied to the test piece 50 changes from the start of the pulling of the test piece to the tensile amount at maximum load. For example, the tensile load to be applied to the test piece 50 from the start of the pulling of the test piece 50 until a predetermined tensile amount (for example, ¾ of the tensile amount at the maximum load) is reached may be less than the 90 N, or the tensile load may reach 90 N or greater during that time.

In terms of adjusting the maximum load that can be applied to the test piece 50 by the tensile test, the total value of the breaking strengths of the element wires 40A, 40B may be adjusted in a case in which the reinforcement layers 24A, 24B are the braids 42. The total value of the breaking strengths of the element wires 40A, 40B is affected by (1) the cross-sectional areas of the element wires 40A, 40B, (2) the number of strands of the element wires of the braid 42 (number of meshes 43 in a cross section orthogonal to the axial direction), and the like. In terms of increasing the breaking strengths, it is advantageous to increase the cross-sectional areas of the element wires 40A, 40B of (1). In terms of increasing the breaking strengths, it is more advantageous to have a greater number of strands of (2). The catheter shaft 10 is preferably configured to have a maximum load (90 N or greater) under the conditions described above by adjustment of any one of (1) and (2). Note that, in a case in which the reinforcement layers 24A, 24B are coils, the maximum load (90 N or greater) under the conditions described above may be obtained by the adjustment of (1).

The maximum load (N) that can be applied to the test piece 50 by the tensile test preferably satisfies the conditions below. The conditions below define the breaking strength required for the catheter shaft 10 as defined in ISO 10555 (general requirements for intravascular catheters supplied in sterile form and intended for single use). With satisfaction of the conditions below, the minimum breaking strength required for the catheter shaft 10 in ISO can be ensured.

### Conditions

When 0.55 mm≤Φ0<0.75 mm: Maximum load is 3 N or greater
When 0.75 mm≤Φ0<1.15 mm: Maximum load is 5 N or greater
When 1.15 mm≤Φ0<1.85 mm: Maximum load is 10 N or greater
When 1.85 mm≤Φ0: Maximum load is 15 N or greater

The catheter shaft 10 preferably satisfies the condition that the reinforcement layers 24A, 24B are not exposed to the outside when application of the tensile load of 90 N to the test piece 50 is started in the tensile test described above. Here, “when application of the tensile load of 90 N to the test piece 50 is started” means when the tensile load of 90 N is first applied in the process of increasing the tensile load to be applied to the test piece 50 in the tensile test. As a result, when the catheter shaft 10 is pulled in a state in which an external force is applied from a blood vessel, as long as the tensile load is within the range (less than 90 N) typically applied by a practitioner, it is possible to avoid a situation in which the reinforcement layers 24A, 24B are exposed to the outside due to partial tearing of the shaft main body 22. In terms of satisfying this condition, the above-described adjustment method adopted to increase the diameter reduction percentage and the elongation percentage without exposing the reinforcement layers 24A, 24B to the outside may be used.

The shaft conditions to be satisfied by the catheter shaft 10 have been described above by using, as an example, the first shaft condition described above in which the diameter reduction percentage to be measured when the elongation percentage of the test piece 50 reaches 100% without breakage of the test piece 50 in the tensile test described above is 5% or greater. Instead, the shaft condition to be satisfied by the catheter shaft 10 may be the second shaft condition described above in which the diameter reduction percentage is 20% or greater when the test piece 50 is pulled until application of the tensile load of 90 N is started in the tensile test described above. In this case, in the tensile test, the tensile test is ended immediately after the pulling until application of the tensile load of 90 N is started. Subsequently, the tensile load to be applied to the test piece 50 is removed, and the diameter reduction percentage to be measured for the test piece 50 removed from the tensile tester need only be 20% or greater. In terms of satisfying the second shaft condition, it is assumed that the test piece 50 is not broken when pulled until application of the tensile load of 90 N is started. In this case as well, preferably the condition that the reinforcement layers 24A, 24B are not exposed to the outside when application of the tensile load of 90 N to the test piece 50 is started is satisfied. Note that, at the time application of the tensile load of 90 N is to be started in the tensile test, the elongation percentage of the test piece 50 exceeds 100%, and is greater than the diameter reduction percentage when the elongation percentage is 100%. From this relationship, the allowable value (20%) of the diameter reduction percentage to be satisfied under the second shaft condition is greater than the allowable value (5%) of the diameter reduction percentage to be satisfied under the first shaft condition.

Next, a tensile test conducted to confirm the above relationship between the diameter reduction percentage and the elongation percentage will be described. In this tensile test, the test pieces 50 of the conditions shown in the following Table 1 were used to conduct a tensile test of pulling until a predetermined elongation percentage was reached. In Table 1, in addition to the types of the test pieces, the ease of elongation of the resin material of the shaft main body, the cross-sectional shape of the element wires, and the number of element wires are described for each type. The cross-sectional shape of the element wires and the cross-sectional shape of the outer reinforcement layer 24B and the inner reinforcement layer 24A are described, in this order. Types A and E are examples in which only the pitch of the element wire was changed.

TABLE 1
Presence

| | | | | | Minimum | Minimum | | | Diameter | Reinforce- | Presence |
|---|---|---|---|---|---|---|---|---|---|---|---|
| | Test | | Element wire | | outer diameter | outer diameter | Elongation | Maximum | reduction | ment | or absence |
| | piece | Shaft main | cross-sectional | No. of | before pulling | after pulling | percentage | load | percentage | layer | of |
| No. | type | body resin | shape | strands | [mm] | [mm] | [%] | [N] | [%] | exposure | breakage |
| 1 | A | Easily | Circular shape / | 16 | 2.96 | 2.90 | 50 | 62 | 2% | None | None |
| 2 | | elongates | Circular shape | (Pitch: | 2.96 | 2.56 | 100 | 61 | 14% | None | None |
| 3 | | | | A) | 2.95 | 2.28 | 150 | 62 | 23% | None | None |
| 4 | | | | | 2.96 | 1.99 | 200 | 64 | 33% | None | None |
| 5 | | | | | 2.95 | 1.87 | 250 | 66 | 37% | None | None |
| 6 | | | | | 2.96 | 1.57 | 500 | 73 | 47% | None | None |
| 7 | | | | | 2.96 | 1.26 | 720 | 90 | 57% | None | None |
| 8 | | | | | 2.96 | 1.06 | 870 | 101 | 64% | Present | Breakage |
| 9 | B | Does not | Flat/Flat | 16 | 2.95 | N/A | 50 | 89 | 0% | Present | Breakage |
| | | easily | | | | | | | | | |
| | | elongate | | | | | | | | | |
| 10 | C | Easily | Flat/ Circular | 16 | 2.97 | 2.89 | 50 | 64 | 3% | None | None |
| 11 | | elongates | shape | | 2.98 | 2.46 | 100 | 62 | 17% | None | None |
| 12 | | | | | 2.97 | 2.30 | 150 | 63 | 23% | None | None |
| 13 | | | | | 2.97 | 2.29 | 200 | 66 | 23% | None | None |
| 14 | | | | | 2.98 | 2.11 | 250 | 75 | 29% | None | None |
| 15 | | | | | 2.98 | 2.03 | 290 | 90 | 32% | None | None |
| 16 | | | | | 2.98 | 1.81 | 550 | 142 | 39% | None | None |
| 17 | | | | | 2.98 | 1.75 | 600 | 162 | 41% | Present | Breakage |
| 18 | D | — | — | — | 2.93 | 2.88 | 50 | 67 | 2% | Present | Breakage |
| 19 | E | Easily | Circular shape / | 16 | 2.96 | 2.94 | 50 | 61 | 1% | None | None |
| 20 | | elongates | Circular shape | (Pitch: | 2.96 | 2.60 | 100 | 61 | 12% | None | None |
| 21 | | | | 3A) | 2.96 | 2.50 | 150 | 62 | 16% | None | None |
| 22 | | | | | 2.96 | 2.37 | 255 | 90 | 20% | None | None |
| 23 | | | | | 2.95 | 2.32 | 290 | 105 | 21% | Present | Breakage |

The test results will now be explained. Table 1 shows, as results of the tensile test for each test piece 50, the maximum load, the presence or absence of exposure of the reinforcement layers 24A, 24B, and the presence or absence of breakage of the test piece 50 in addition to the minimum outer diameter before pulling, the minimum outer diameter after pulling, the elongation percentage, and the diameter reduction percentage. The maximum load in Table 1 indicates the maximum load applied from the start of the pulling of the test piece 50 until a predetermined elongation percentage was reached. No. 9 (type B) and No. 18 (type D) are examples in which breakage occurred after the start of pulling and before the pulling until the elongation percentage reached 50%.

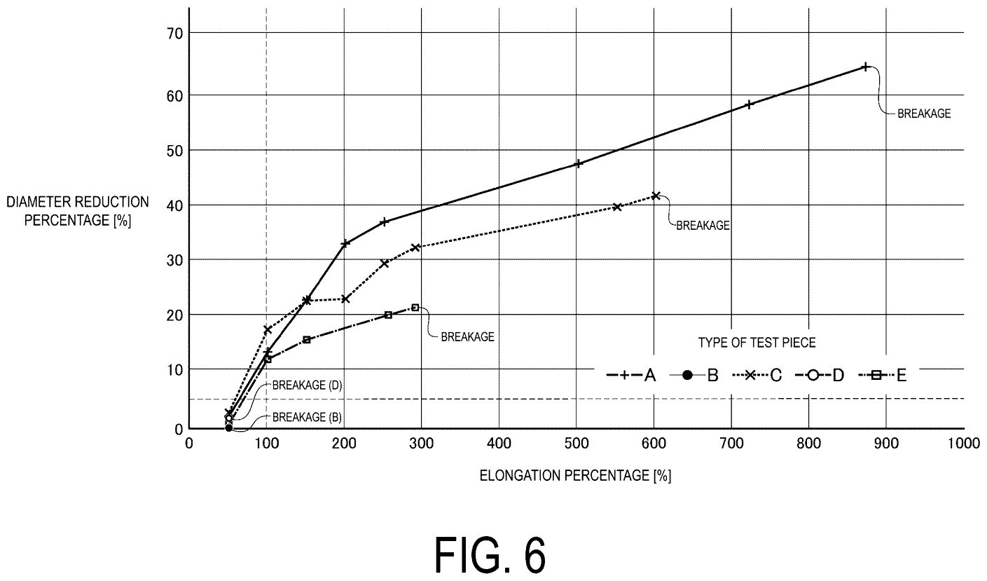

FIG. 6 is a graph showing the results of the tensile test described above. Types B and D are examples in which breakage occurs when the maximum elongation percentage is less than 50%, and the diameter reduction percentage fails to reach 5% or greater when the elongation percentage reaches 100% without breakage of the test piece. On the other hand, types A, C, and E are examples in which the diameter reduction percentage is 5% or greater when the elongation percentage is 100% without breakage of the test piece. From these comparisons, it can be understood that these were advantageous in terms of setting the diameter reduction percentage to 5% or greater when the elongation percentage reaches 100% without breakage of the test piece 50. In addition, it can be understood from FIG. 6 that there exists an advantageous tendency that, as the elongation percentage acquirable without breakage of the test piece 50 increases, the diameter reduction percentage is increased. For example, when type E having an acquirable elongation percentage of the test piece 50 of less than 300% is compared with types A and C having elongation percentages of 300% or greater, it can be understood that the acquirable diameter reduction percentages of the latter are greater.

In addition, from the comparison between types A and E, it can be understood that, as the pitches of the element wires narrowed in the order of E→A, the results were more advantageous in terms of increasing the elongation percentage and the diameter reduction percentage acquirable. From the comparison of types A and C, it can be understood that the circular cross section of the element wire is more advantageous than the flat cross section in terms of increasing the elongation percentage and the diameter reduction percentage acquirable.

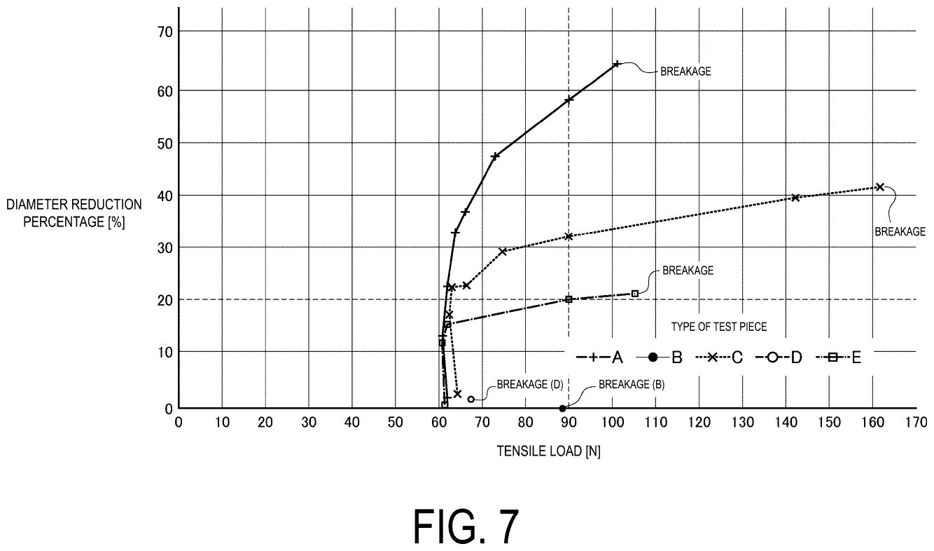

FIG. 7 is a graph in which the results shown in Table 1 are arranged in terms of the relationship between the tensile load and the diameter reduction percentage. Types B and D are examples in which the test piece broke before the tensile load of 90 N was applied to the test piece, and the diameter reduction percentage when application of the tensile load of 90 N to the test piece was started failed to reach 20% or greater. Types A, C, and E are examples in which the diameter reduction percentage when application of the tensile load of 90 N to the test piece was started is 20% or greater.

The above embodiments are illustrative. Technical ideas obtained by being abstracted should not be interpreted as limited to the contents of the embodiments. Numerous design changes, such as variations, addition, and deletion of components, can be made to the contents of the embodiments. In the embodiments described above, the content in which such design changes can be made has been emphasized with expressions such as of “the embodiment”. However, design changes are also possible even in the content without such an expression. Hatching in sections of the drawings does not limit the material of a hatched object.

The maximum diameter reduction percentage and elongation percentage of the test piece 50 that can be acquired in a state in which the reinforcement layers 24A, 24B are not exposed may be measured according to the following procedure.
(1) First, conduct a first tensile test of pulling the test piece 50 until it breaks. In the first tensile test, measure the elongation percentage of the test piece 50 when the test piece 50 is broken.
(2) Next, conduct a second tensile test using the test piece 50 obtained from the same sample as the test piece 50 used in the first tensile test. In the second tensile test, pull the test piece 50 until the elongation percentage becomes the elongation percentage measured in the first tensile test minus 10%. Subsequently, determine whether the reinforcement layer of the test piece 50 obtained by this tensile test is exposed.
(3) In a case in which the reinforcement layer is exposed in (2), conduct a tensile test of pulling until the elongation percentage reaches the elongation percentage reached in the previous tensile test minus 10%, and repeat the series of steps until the presence or absence of exposure of the reinforcement layer of the test piece 50 obtained by the tensile test is determined. Repeat this series of steps until the test piece 50 in which the reinforcement layer is not exposed after the tensile test is obtained.
(4) Once the test piece 50 in which the reinforcement layer is not exposed after the tensile test is obtained in (2), (3), measure the diameter reduction percentage of that test piece 50. The diameter reduction percentage of the test piece 50 measured in this manner and the elongation percentage reached in the tensile test for the test piece 50 may be used as the maximum diameter reduction percentage and elongation percentage of the test piece 50 that can be acquired in a state in which the reinforcement layer is not exposed.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A catheter shaft comprising:
a shaft main body having a multi-lumen structure; and
at least one reinforcement layer embedded in the shaft main body, wherein
when, during a tensile test of pulling a test piece obtained from the catheter shaft under test conditions of a 30-mm distance between chucks and a tensile speed of 300 mm/min, a percentage of change amount in a minimum outer diameter of the test piece after pulling to the minimum outer diameter before pulling at a predetermined position of the test piece is defined as a diameter reduction percentage (%), and a percentage of change amount in an axial-direction dimension of the catheter shaft after pulling to the axial-direction dimension before pulling is defined as an elongation percentage (%), the diameter reduction percentage is 5% or greater when the elongation percentage reaches 100% without breakage of the test piece in the tensile test.

2. The catheter shaft according to claim 1, wherein
the at least one reinforcement layer is not exposed to the outside when the elongation percentage reaches 100% in the tensile test.

3. The catheter shaft according to claim 1, wherein
the at least one reinforcement layer is a braid formed by braiding a plurality of element wires into a tubular and net-like shape, and
a factor affecting an ease of movement of the plurality of element wires when the braid is about to undergo elongation deformation is adjusted so that the elongation percentage is 100% or greater.

4. The catheter shaft according to claim 1, wherein
a maximum tensile load (N) that is applicable to the test piece until the test piece breaks in the tensile test is 90 N or greater.

5. The catheter shaft according to claim 1, wherein
the at least one reinforcement layer is not exposed to the outside when application of a tensile load of 90 N to the test piece is started in the tensile test.

6. A catheter shaft comprising:
a shaft main body having a multi-lumen structure; and
at least one reinforcement layer embedded in the shaft main body, wherein
when, during a tensile test of pulling a test piece obtained from the catheter shaft under test conditions of a 30-mm distance between chucks and a tensile speed of 300 mm/min, a percentage of change amount in a minimum outer diameter of the test piece after pulling to the minimum outer diameter before pulling at a predetermined position of the test piece is defined as a diameter reduction percentage (%), the diameter reduction percentage is 20% or greater when application of a tensile load of 90 N to the test piece is started in the tensile test.

7. A catheter device comprising:
the catheter shaft according to claim 1.
